(19) United States
(12) Plant Patent Application Publication
(10) Pub. No.: US 2003/0041361 P1
(43) Pub. Date: Feb. 27, 2003

(54) Floribunda rose plant named 'Meichibon'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 09/933,176

(22) Filed: Aug. 21, 2001

(51) Int. Cl. A01H 5/00

(52) U.S. Cl. PLT/145

(57) ABSTRACT

A new and distinct variety of Floribunda rose plant is provided which very abundantly forms attractive very pale yellow blossoms. Such blossoms possess no fragrance. The plant exhibits a vigorous bushy growth habit, forms very dense medium green and semi-glossy foliage, and possesses very good disease resistance. The new variety is particularly well suited for providing colorful ornamentation in the landscape.

### SUMMARY OF THE INVENTION

The new variety of Rosa hybrida Floribunda rose plant was created by artificial pollination wherein two parents were crossed which previously had been studied in the hope that they would contribute the desired characteristics. The female parent (i.e., the seed parent) of the new variety was the product of the cross of the ‘Meibaltaz’ variety (U.S. Plant Pat. No. 8,499) and the ‘Landora’ variety (non- patented in the United States). The male parent (i.e., the pollen parent) was the ‘Delge’ variety (non-patented in the United States). The parentage of the new variety can be summarized as follows:

(‘Meibaltaz’×‘Landora’)×‘Delge’.

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) forms on a very abundant basis attractive double very pale yellow blossoms,

(b) exhibits a vigorous bushy growth habit,

(c) displays very good disease resistance,

(d) forms attractive very dense medium green semi-glossy foliage, and

(e) is particularly well suited for growing as ornamentation in parks and gardens.

The new variety well meets the needs of the horticultural industry and is particularly well suited for growing in the landscape.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meichibon’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during June while budded on Rosa froebelli understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the sepals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface

FIG. 15—illustrates a specimen of a leaf with five leaflets—plan view—under surface; and

FIG. 16—illustrates a leaf with seven leaflets—plan view—upper surface.

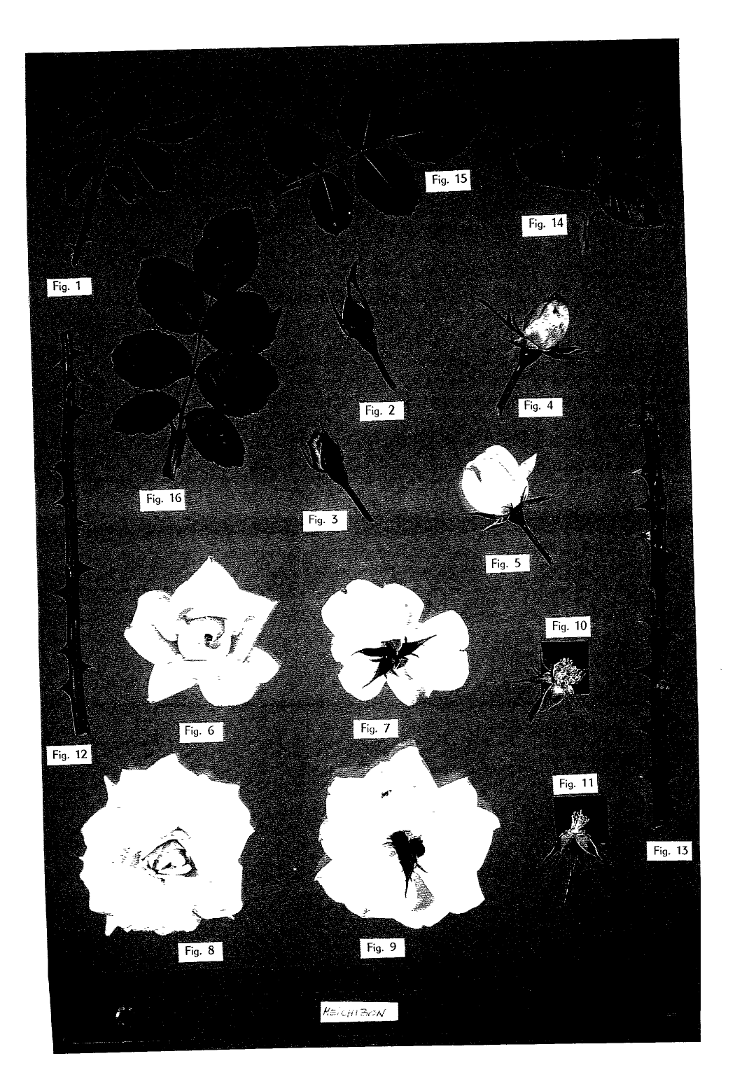

### DETAILED DESCRIPTION

The chart used in the identification of the colors is that of The Royal Horticultural Society (R.H.S. Colour Chart). The description is based on the observation of two year-old plants during September while budded on Rosa froebelli understock and growing outdoors at Le Cannet des Maures, Var, France. The coloration in common terms precedes reference to the chart in some instances.

Class: Floribunda.

Plant:

Height.—approximately 90 cm on average at the end of the growing season.

Width.—approximately 70 to 90 cm on average at the end of the growing season.

Habit.—bushy.

Branches:

Color.—young stems: near Yellow-Green Group 146A. adult wood: near Yellow-Green Group 146C.

Thorns.—size: medium (as illustrated). quantity: moderately numerous (as illustrated). color: near Greyed-Orange Group 165A and 165B. shape: rather flat on upper surface and slightly concave on the under surface.

Leaves:

Stipules.—adnate, pectinate, and rather broad.

Petioles.—upper surface: near Green Group 139A in coloration. under surface: near Yellow-Green Group 146B in coloration.

Leaflets.—number: 3, 5, and 7. shape: ovate. serration: regular (as illustrated). texture: consistent and rather firm. general appearance: very dense, medium green, and semi-glossy foliage. color (young foliage): upper surface: near Green Group 139A. under surface: near Yellow-Green Group 146A. color (adult foliage): upper surface: near Green Group 139A. under surface: near Yellow-Green Group 146A.

Inflorescence:

Number of flowers.—usually 2 to 5 flowers per stem.

Peduncle.—near Yellow-Green Group 146A in coloration, medium in size, and approximately 2.5 to 3 cm in length.

Sepals.—upper surface: near Yellow-Green Group 148B and 148C in coloration. under surface: near Yellow-Green Group 146C in coloration. configuration: elongated and commonly with some weak extensions (as illustrated).

Buds.—shape: conical. size: medium. length: approximately 2 cm on average. color upon opening: upper surface: Primrose Yellow, near Yellow Group 4D, with a very light edging on the internal petals of Red Group 38D. under surface: Primrose Yellow, near Yellow Group 4D, with a very light edging on the internal petals of Red Group 38D.

Flower.—shape: double and cup-shaped. diameter: commonly approximately 8 to 10 cm on average. color (when opening begins): upper surface: Primrose Yellow, near Yellow Group 4D, with a very light edging on the internal petals of Orient Pink, Red Group 36A. under surface: Primrose Yellow, near Yellow Group 4D, with a very light edging on the internal petals of Orient Pink, Red Group 36A. color (when blooming): upper surface: Primrose Yellow, near Yellow Group 4D, with a very light edging on the internal petals of Orient Pink, Red Group 36A. under surface: Primrose Yellow, near Yellow Group 4D, with a very light edging on the internal petals of Orient Pink, Red Group 36A. color (at end of opening): upper surface: Primrose Yellow, near Yellow Group 4D, with a very light edging on the internal petals of Orient Pink, Red Group 36A. under surface: Primrose Yellow, near Yellow Group 4D, with a very light edging on the internal petals of Orient Pink, Red Group 36A. fragrance: none. lasting quality: very long. petal shape: with a cordate base and a reflexed tip. petal number: approximately 99 to 107 on average. stamen: approximately 57 on average. anthers: near Yellow-Orange Group 15B in coloration. filaments: near Yellow-Orange Group 15D in coloration. pistils: approximately 66 on average. stigmas: near Yellow-Orange Group 15D in coloration. style: near Yellow-Orange Group 15D in coloration. receptacle: near Yellow-Green Group 146A in coloration, and in longitudinal section in the shape of a funnel.

Development:

Vegetation.—very strong.

Blooming.—very abundant.

Resistance to diseases.—very good in general.

Aptitude to bear fruit.—medium.

I claim:

1. A new and distinct variety of Floribunda rose plant characterized by the following combination of characteristics:
(a) forms on a very abundant basis attractive double very pale yellow blossoms,
(b) exhibits a vigorous bushy growth habit,
(c) displays very good disease resistance,
(d) forms attractive very dense medium green semi-glossy foliage, and
(e) is particularly well suited for growing in parks and gardens;
substantially as herein shown and described.
